(19) United States
(12) Patent Application Publication
(10) Pub. No.: US 2010/0028890 A1
(43) Pub. Date: Feb. 4, 2010

(54) COMPOSITIONS, METHODS, AND KITS FOR ANALYZING DNA METHYLATION

(75) Inventor: Victoria L. BOYD, San Carlos, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/498,314

(22) Filed: Jul. 6, 2009

Related U.S. Application Data
(63) Continuation of application No. 11/352,143, filed on Feb. 9, 2006, now abandoned.
(60) Provisional application No. 60/654,162, filed on Feb. 18, 2005.

(51) Int. Cl. C12Q 1/68; C12P 19/34

(52) U.S. Cl. 435/6; 435/91.2

(57) ABSTRACT

Compositions, methods, and kits for reducing strand amplification bias using bisulfite treated gDNA are provided. Methods for detecting and for quantitating the amplified bisulfite treated gDNA and inferring the presence, absence, and/or degree of methylation of target cytosine(s) in the gDNA are also provided. Such methods typically employ tailed first primer pairs, which can, but need not comprise nucleotide analogs, and optionally second primer pairs.

### CROSS-REFERENCE TO RELATED APPLICATIONS

This application is a continuation of U.S. patent application Ser. No. 11/352,143, filed Feb. 9, 2006 (pending), which claims the benefit of priority under 35 U.S.C. §119(e) to U.S. Patent Application No. 60/654,162, filed Feb. 18, 2005, both of which are incorporated herein by reference in their entireties.

### FIELD

The present teachings generally relate to compositions, methods, and kits for amplifying bisulfite treated genomic DNA (gDNA) with reduced strand amplification bias and for detecting and for quantitating the presence, absence, and/or degree of gDNA methylation.

### INTRODUCTION

The methylation of cytosine residues in DNA (technically cytidine residues) is an important epigenetic alteration in eukaryotes. In humans and other mammals methylcytosine is found almost exclusively in cytosine-guanine (CpG) dinucleotides. DNA methylation plays an important role in gene regulation and changes in methylation patterns are reportedly involved in many human cancers and certain human diseases. Among the earliest and most common genetic alterations observed in human malignancies is the aberrant methylation of CpG islands, particularly CpG islands located within the 5′ regulatory regions of genes, causing alterations in the expression of such genes. Subsequently, there is great interest in using DNA methylation markers as diagnostic indicators for early detection, risk assessment, therapeutic evaluation, recurrence monitoring, and the like (see, Widschwendter et al., Clin. Cancer Res. 10:565-71, 2004; Dulaimi et al., Clin. Cancer Res. 10:1887-93, 2004; Topaloglu et al., Clin. Cancer Res. 10:2284-88, 2004; Laird, Nature Reviews, 3:253-266, 2003; Fraga et al., BioTechniques 33:632-49, 2002; Adorjan et al., Nucleic Acids Res. 30(5):e21, 2002; and Colella et al., BioTechniques, 35(1):146-150, 2003). There is also great scientific interest in the role of DNA methylation in embryogenesis, cellular differentiation, transgene expression, transcriptional regulation, and maintenance methylation, among other things.

Bisulfite genomic sequencing is a widely used method for evaluating methylation patterns in gDNA, including the relative methylation level of individual cytosines of interest. Typically, gDNA is treated with sodium bisulfite which converts cytosine bases to uracil, while methylated cytosines are generally nonreactive. The bisulfite treated gDNA target sequence is then PCR amplified using sequence specific primers to yield sequences in which uracil residues are converted to thymine, while methylated cytosine is amplified as cytosine. In the case of samples comprising mixed cell populations, for example but not limited to tumor biopsy samples containing both normal cells and cancerous cells, the cytosine content of the amplified DNA from the various cell subpopulations can be very different, with unmethylated DNA being T-rich and C-deficient after conversion, while fully methylated DNA can retain at least some of its original cytosine content. This content disparity can lead to difficulties in quantitating the relative degree of methylation, including primer design problems and strand amplification bias, i.e., the preferential amplification of T-rich sequences and a corresponding under-representation of the methylated sequences in the resulting amplification products. In addition to problems of increased secondary structure associated with the G-C rich methylated strand, other potential problems encountered when amplifying bisulfite treated gDNA that could result in lack of quantitative amplification include mispriming, resulting in non-specific amplification of non-target sequences, and other amplification artifacts such as primer dimer formation. As a result, the degree of methylation of a particular cytosine residue within a gDNA sequence can oftentimes not be accurately determined. See, e.g., Warnecke et al., Nucl. Acids Res. 25:4422-4426, 1997; Voss et al., Anal. Chem. 70:3813-23, 1998; and Tusnady et al., Nucl. Acids Res. 33:e9, 2005.

### SUMMARY

Certain of the present teachings are directed to compositions, methods, and kits for: amplifying bisulfite treated gDNA while reducing strand amplification bias; detecting the extension products generated from bisulfite treated gDNA and inferring the presence or absence of methylated cytosine residues in the gDNA; and quantitating the extension products to determine the degree of cytosine methylation. According to the instant teachings, amplification bias of bisulfite converted gDNA is decreased by using tailed primer pairs that become incorporated into the disclosed extension products; increasing reaction temperatures, including without limitation reaction temperature shifts; using nucleotide analogs, including without limitation chemically incorporated analogs and/or enzymatically incorporated analogs; or combinations thereof.

Certain disclosed methods comprise a tailed first primer pair and, in some embodiments, a second primer pair. Typically, a first primer pair is designed to anneal with regions of the bisulfite treated gDNA that flank the sequence comprising the target cytosines to be evaluated, for example but not limited to, a promoter region for a tumor suppressor gene or other CpG island of interest. In some embodiments, a tailed first primer of the first primer pair acts as a reverse primer and primes the synthesis of a first extension product on a bisulfite treated gDNA template. In some embodiments, a tailed second primer of the first primer pair acts as a forward primer and primes the synthesis of a second extension product using the first extension product as a template. In some embodiments, additional tailed first primers are used to generate third extension products using a second extension product as the template. In some embodiments, additional tailed second primers are used to generate second extension products using a third extension product as a template. In other embodiments, a second primer pair is used to generate additional second extension products and third extension products using the second and third extension products as templates. Some embodiments comprise a multiplicity of different first primer pairs for amplifying a multiplicity of different bisulfite treated gDNA target sequences. Some embodiments comprise a different second primer pair for each bisulfite treated gDNA target sequence being amplified; in some embodiments, one second primer pair is used with at least two different bisulfite treated gDNA target sequences being amplified.

In some embodiments, a bisulfite treated gDNA target sequence is amplified using a tailed first primer pair and optionally, a second primer pair. The first primer pair comprises a tailed first primer and a corresponding tailed second primer, wherein (1) the first primer comprises (a) a target-complementary portion and (b) a tail comprising a first primer-binding site upstream of the target-complementary portion and (2) the second primer comprises (a) a first extension product-complementary portion and (b) a tail comprising a second primer-binding site upstream of the first extension product-complementary portion. The target-complementary portion of the first primer is designed to hybridize with a first region of the bisulfite treated gDNA flanking the target sequence and participate in “first strand synthesis”. The first extension product-complementary portion of the second primer is the same as or substantially the same as a second region of the target sequence to allow the second primer to hybridize with the first extension product.

Typically, a tailed first primer hybridizes with a region of bisulfite treated gDNA, typically downstream of the corresponding target sequence, and under suitable conditions the hybridized first primer is extended to generate a first extension product that includes the incorporated tailed first primer. A corresponding tailed second primer hybridizes with the first extension product, typically downstream of the complement of the target sequence, and under suitable conditions is extended to generate a second extension product that includes the incorporated tailed second primer and the complement of the tailed first primer.

In some embodiments, another tailed first primer anneals with the second extension product and is extended to generate a third extension product that includes the complement of the tailed second primer. In some embodiments, another tailed second primer anneals with the third extension product and is extended to generate an additional second extension product. In certain embodiments, the steps of (1) annealing the tailed first primer and/or tailed second primer, (2) extending the annealed tailed first primer and/or tailed second primer to generate a third or an additional second extension product, and (3) denaturing the resulting extension product duplexes are repeated one or more times using a multiplicity of tailed first primer pairs.

In some embodiments, a tailed first primer, a tailed second primer, or a tailed first primer and a tailed second primer comprise a nucleotide analog. Typically, the nucleotide analog(s) in the tailed first and/or tailed second primer is selected to increase the annealing temperature of the primer, but not always. In some embodiments, a nucleotide analog is enzymatically incorporated into an extension product during primer extension. In some embodiments, a tailed first primer and/or a tailed second primer comprise a multiplicity of nucleotide analogs. In some embodiments, an extension product comprises a multiplicity of nucleotide analogs.

Some embodiments of the disclosed methods further comprise a second primer pair that includes a third primer and a fourth primer. The third primer is designed to anneal with the complement of the first primer-binding site in the second extension product and can be extended to generate a third extension product. The fourth primer is designed to anneal with the complement of the second primer-binding site in the third extension product and can be extended to generate an additional second extension product. In certain embodiments, the steps of (1) annealing the third and/or fourth primers, (2) extending the annealed third and/or fourth primers to generate a third or an additional second extension product, and (3) denaturing the resulting extension product duplexes are repeated one or more times using a multiplicity of second primer pairs.

According to certain methods, a first extension product, a second extension product, a third extension product, a surrogate of an extension product, or combinations thereof, are detected. In some embodiments, such detection comprises quantitating the extension products or their surrogates and inferring the degree of methylation of one or more cytosine residues in the corresponding target sequence.

In some embodiments, at least two versions of a target locus with slightly differing nucleotide composition are being evaluated, for example but not limited to two targets comprising alternate alleles of certain single nucleotide polymorphisms (“SNPs”) wherein one allele has an A:T pair and the second allele has a G:C pair at the SNP site. Those in the art will appreciate that the compositions and methods of the current teachings may allow such target loci to be PCR amplified with reduced bias. In some embodiments, gDNA is not bisulfite treated prior to amplifying the gDNA.

In some embodiments, kits are disclosed to expedite performance of one or more of the disclosed methods. These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIGS. 1A and 1B: depict an exemplary embodiment of a disclosed amplification method. Exemplary target cytosines are indicated by “?”.

FIG. 2: depicts an electropherogram showing the nucleotide sequence obtained according to one exemplary detection method, described in Example 2.

FIG. 3: depicts an electropherogram showing the nucleotide sequence obtained according to one exemplary detection method, described in Example 3.

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. For example, “a tailed first primer” means that more than one tailed first primer can be present; for example, one or more copies of a particular tailed first primer species, as well as one or more species of tailed first primer, such as a tailed first primer that hybridizes with a particular region of bisulfite treated gDNA that flanks a target sequence and a different tailed first primer that hybridizes with another region of bisulfite treated gDNA that flanks a different target sequence. Also, the use of “comprise”, “comprises”, “comprising”, “contain”, “contains”, “containing”, “include”, “includes”, and “including” are not intended to be limiting.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials differs from or contradicts this application, including but not limited to defined terms, term usage, described techniques, or the like, this application controls.

### I. DEFINITIONS

The term “affinity tag” as used herein refers to a component of a multi-component complex, wherein the components of the multi-component complex specifically interact with or bind to each other. Exemplary multiple-component affinity tag complexes include without limitation, ligands and their receptors, for example but not limited to, avidin-biotin, streptavidin-biotin, and derivatives of biotin, streptavidin, or avidin, including without limitation, 2-iminobiotin, desthiobiotin, NeutrAvidin (Molecular Probes, Eugene, Oreg.), CaptAvidin (Molecular Probes), and the like; binding proteins/peptides and their binding partners, epitope tags and their corresponding anti-epitope antibodies; haptens, for example but not limited to dinitrophenol (“DNP”) and digoxigenin (“DIG”), and their corresponding antibodies; aptamers and their binding partners; fluorophores and their corresponding anti-fluorophore antibodies; and the like. In certain embodiments, affinity tags are part of a separating means, part of a detecting means, or both.

The term “or combinations thereof” as used herein refers to all permutations and combinations of the listed items preceding the term. For example, “A, B, C, or combinations thereof” is intended to include at least one of: A, B, C, AB, AC, BC, BAC, or ABC, and if order is important in a particular context, also BA, CA, CB, CBA, BCA, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term refers. For example, a tailed first primer of a particular first primer pair corresponds to the tailed second primer of the same first primer pair, and vice versa. A third primer is designed to anneal with the complement of the first primer-binding portion of the second extension product and the corresponding fourth primer of the same second primer pair is designed to anneal with the complement of the second-primer binding portion of the third extension product. A particular affinity tag binds to the corresponding affinity tag, for example but not limited to, biotin binding to streptavidin. A particular hybridization tag anneals with its corresponding hybridization tag complement; and so forth.

The terms “hybridizing” and “annealing”, including variations of these terms such as annealed, hybridization, anneal, hybridizes, and so forth, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and tailed primers anneal to complementary or substantially complementary regions of gDNA or extension products are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349, 1968. In general, whether such annealing takes place is influenced by, among other things, the length of the hybridizing region of the primers and their complementary sequences, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or groove binders in the primer or reporter probe can also influence hybridization conditions. Thus, the preferred annealing conditions will depend upon the particular application. Such conditions, however, can be routinely determined by persons of ordinary skill in the art, without undue experimentation. Typically, annealing conditions are selected to allow complementary or substantially complementary portions of primers, hybridization tags, reporter probes, and the like, to selectively hybridize with their corresponding target sequence, extension product, hybridization tag complement, reporter probe binding portion, respectively, but not hybridize to any significant degree to other sequences in the reaction, e.g., mispriming and primer dimer formation.

The term “hybridization tag” as used herein refers to an oligonucleotide sequence that can be used for: separating the element (e.g., tailed primers, third primers, fourth primers, first extension products, second extension products, third extension products, or extension product surrogates, including without limitation, ZipChute™ reagents, etc.) of which it is a component or to which it is hybridized, including without limitation, bulk separation; tethering or attaching the element to which it is bound to a capture surface, which may include separating and/or detecting; annealing a corresponding hybridization tag complement; or combinations thereof. In certain embodiments, the same hybridization tag is used with a multiplicity of different elements to effect bulk separation or capture surface attachment. In certain embodiments, a hybridization tag provides a unique “address” or identifier to the element containing the hybridization tag. In certain embodiments, this address can be used to identify the corresponding element, for example but not limited to, hybridizing to a particular address or position on an ordered capture surface, including without limitation, a microarray or a bead array comprising a corresponding hybridization tag complement. In certain embodiments, a primer comprises a unique hybridization tag that is incorporated into an extension product so that the hybridization tag can serve as a reporter probe-binding site, used to bind a reporter probe for detecting that extension product or its surrogate (see, e.g., U.S. Pat. No. 6,270,967). A “hybridization tag complement” typically refers to an oligonucleotide that comprises a nucleotide sequence that is complementary to at least part of the corresponding hybridization tag. In various embodiments, hybridization tag complements serve as capture moieties for attaching a hybridization tag:element complex to a capture surface for identification, such as multiplex decoding on a microarray or serve as “pull-out” sequences for bulk separation procedures. In certain embodiments, a hybridization tag complement comprises a reporter group, a mobility modifier, an affinity tag, a reporter probe-binding site, or combinations thereof. In certain embodiments, a hybridization tag complement is annealed to a corresponding hybridization tag and, subsequently, at least part of that hybridization tag complement is released and detected. In certain embodiments, detecting comprises a reporter group on or attached to a hybridization tag complement or at least part of a hybridization tag complement.

Typically, hybridization tags and their corresponding hybridization tag complements are selected to minimize: internal self-hybridization; and cross-hybridization with different hybridization tag species, nucleotide sequences in a sample or reaction composition, including but not limited to target or background sequences, different species of hybridization tag complements, sequence-specific portions of primers, and the like; but should be amenable to facile hybridization between the hybridization tag and its corresponding hybridization tag complement. Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags can be found in, among other places, U.S. Pat. Nos. 6,309,829 (referred to as “tag segment” therein); 6,451,525 (referred to as “tag segment” therein); 6,309,829 (referred to as “tag segment” therein); 5,981,176 (referred to as “grid oligonucleotides” therein); 5,935,793 (referred to as “identifier tags” therein); and PCT Publication No. WO 01/92579 (referred to as “addressable support-specific sequences” therein); and Gerry et al., J. Mol. Biol. 292:251-262, 1999) (referred to as “zip-codes” and “zip-code complements” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a primer, an extension product, or both; or they can be located internally. In certain embodiments, a hybridization tag is attached to a primer, an extension product, a reporter probe, or combinations thereof, via a linker arm. In certain embodiments, the linker arm is cleavable.

In certain embodiments, hybridization tags are at least 12 nucleotide bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a ΔTₘ range (Tₘₐₓ-Tₘᵢₙ) of no more than 10° C. of each other. In certain embodiments, at least two hybridization tag:hybridization tag complement duplexes have melting temperatures that fall within a ΔTₘ range of 5° C. or less of each other.

The term “reporter group” is used in a broad sense herein and refers to any identifiable tag, label, or moiety. The skilled artisan will appreciate that many different species of reporter groups can be used in the present teachings, either individually or in combination with one or more different reporter group. In certain embodiments, a reporter group emits a fluorescent, a chemiluminescent, a bioluminescent, a phosphorescent, a radioactive, a calorimetric, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, electrochemiluminescence labels, and mass spectrometer compatible reporter groups, such as mass tags, charge tags, and isotopes.

The term reporter group also encompasses an element of multi-element indirect reporter systems, including without limitation, affinity tags such as biotin:avidin, antibody:antigen, and the like, in which one element interacts with one or more other elements of the system in order to effect the potential for a detectable signal. Exemplary multi-element reporter systems include an oligonucleotide comprising biotin and a streptavidin-conjugated fluorophore, or vice versa; an oligonucleotide comprising a DNP reporter group and a fluorophore-labeled anti-DNP antibody; and the like. In certain embodiments, reporter groups, particularly multi-element reporter groups, are not necessarily used for detection, but serve as affinity tags for isolation/separation, for example but not limited to, a biotin reporter group and a streptavidin-coated capture surface, or vice versa; a DIG reporter group and a capture surface comprising an anti-DIG antibody or a DIG-binding aptamer; a DNP reporter group and a capture surface comprising an anti-DNP antibody or a DNP-binding aptamer; and the like. Detailed protocols for attaching reporter groups to nucleic acids can be found in, among other places, G. T. Hermanson, Bioconjugate Techniques, Academic Press, San Diego, 1996; Current Protocols in Nucleic Acid Chemistry, S. L. Beaucage et al., eds., John Wiley & Sons, New York, N.Y. (2000), including supplements (“Beaucage”); Haugland, Handbook of Fluorescent Probes and Research Products, 9^{th} ed., Molecular Probes, 2002; and Pierce Applications Handbook and Catalog 2003-2004, Pierce Biotechnology, Rockford, Ill., 2003.

Multi-element interacting reporter groups are also within the scope of the term reporter group, such as fluorophore-quencher pairs, including without limitation fluorescent quenchers and dark quenchers (also known as non-fluorescent quenchers). A “fluorescent quencher” can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A “dark quencher”, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorophore-quencher pairs can emit fluorescence when the members of the pair are physically separated, for example but without limitation, nuclease probes such as TaqMan® probes. Other dual-labeled probes comprising fluorophore-quencher pairs can emit fluorescence when the members of the pair are spatially separated, for example but not limited to hybridization probes such as molecular beacons or extension probes such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

The term “surrogate” as used herein refers to any molecule or moiety whose detection or identification indicates the existence of a corresponding bisulfite treated gDNA target sequence including a particular target cytosine in a gDNA sequence and in some embodiments, allows the presence, absence, or degree of target cytosine methylation to be inferred. Exemplary surrogates include but are not limited to, a first extension product, a second extension product, a third extension product, or portions thereof; moieties cleaved or released from an extension product or an extension product surrogate; sequencing fragments generated from an extension product or a sequence within an extension product; complementary strands or counterparts of an extension product or extension product surrogate; reporter probes that are or were annealed to a extension product or another extension product surrogate, including but not limited to cleavage and extension products thereof, such as a cleavage fragment of a TaqMan® probe or the product of a scorpion primer; hybridization tag complements that are or were annealed to an extension product or another extension product surrogate, including but not limited to ZipChute™ reagents (typically a molecule or complex comprising a hybridization tag complement, a mobility modifier, and a reporter group, generally a fluorescent reporter group; see, e.g., Applied Biosystems Part Number 4344467 Rev. C; see also U.S. Provisional Patent Application Ser. No. 60/517,470, now U.S. patent application Ser. No. 10/982,619) or parts of hybridization tag complements; detectable luminescence or color from a chemical and/or enzymatic reaction; and the like. It is to be understood that a third extension product can serve as a surrogate for the corresponding first extension product and/or second extension product, and the corresponding bisulfite treated gDNA target sequence; that a second extension product can serve as a surrogate for the corresponding first extension product and corresponding bisulfite treated gDNA target sequence; and that a first extension product can serve as a surrogate for the corresponding bisulfite treated gDNA target sequence. Thus, the detection of any of these surrogates, either directly or indirectly, allows the presence, absence, or degree of methylation of a particular target cytosine in the gDNA target sequence to be inferred.

The terms “Tm” and “melting temperature” are used interchangeably and refer to the temperature at which a population of double-stranded nucleic acid molecules, including without limitation, a first extension product-bisulfite treated gDNA duplex, a first extension product-second extension product duplex, and a second extension product-third extension product duplex, become half (50%) dissociated. The correlate, “Tm anneal” or “annealing temperature”, which refers to the temperature at which 50% of a population of primers anneal with their complementary sequence, for example but not limited to, a tailed first primer annealed to the complementary region of the corresponding bisulfite treated gDNA or the corresponding second extension product, the tailed second primer annealed to the corresponding first extension product or third extension product, the third primer annealed to the corresponding second extension product, and the fourth primer annealed with the corresponding third extension product, is also within the intended scope of the term Tm as used herein.

Several formulas and computer algorithms for calculating Tm, including chimeric oligomers comprising conventional nucleotides and nucleotide analogs, are well-known in the art. According to one such predictive formula for oligonucleotides, Tm=(4×number of G+C)+(2×number of A+T). The Tm for a particular oligonucleotide, such as a probe or primer, can also be routinely determined using known methods, without undue experimentation. Descriptions of Tm/melting temperatures and their calculation can be found in, among other places, The Nucleic Acids Protocols Handbook, Rapley, ed., Humana Press, 2000 (“Rapley”); Nielsen, Exiqon Technical Note LNA Feb. 7, 2002, Exiqon A/S; McPherson and Moller, PCR: The Basics, Bios Scientific Publishers, 2000 (“McPherson”); Finn et al., Nucl. Acids Res. 17:3357-63, 1996; and on the internet at, among other places, “appliedbiosystems.com/support/techtools/calc/”, “207.32.43.70/biotools/oligocalc/oligocalc.asp”, and “www-structure.llnl.gov/MB_elves/tmcalc.html”.

The term “mobility-dependent analytical technique” as used herein, refers to any means for separating different molecular species based on differential rates of migration of those different molecular species in one or more separation techniques. Exemplary mobility-dependent analytical techniques include electrophoresis, chromatography, sedimentation, e.g., gradient centrifugation, field-flow fractionation, multi-stage extraction techniques, and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

The term “mobility modifier” as used herein refers to a molecular entity, for example but not limited to, a polymer chain, that when added to an element (e.g., a primer, including tailed first primers, tailed second primers, third primers, and/or fourth primers, an extension product, a hybridization tag, a hybridization tag complement, or combinations thereof) affects the mobility of the element to which it is hybridized or bound, covalently or non-covalently, in a mobility-dependent analytical technique. In some embodiments, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543; Grossman et al., Nucl. Acids Res. 22:4527-34, 1994). In certain embodiments, a multiplicity of different primers or extension products that do not comprise mobility modifiers have the same or substantially the same mobility in a mobility-dependent analytical technique. Typically, such primers or extension products can be separated or substantially separated in a mobility-dependent analytical technique when each such species further comprises an appropriate mobility modifier. Descriptions of mobility modifiers and their use can be found in, among other places, PCT Publication No. WO 01/92579.

The term “nucleotide analogs” refers to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH₄⁺, Na⁺, if such counterions are present. Exemplary modified nucleotide base portions include but are not limited to 5-methylcytosine (5mC); C-5-propynyl analogs, including but not limited to, C-5 propynyl-C and C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30, 1998), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chem., 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles. In one class of nucleotide analogs, known as peptide nucleic acids, including pseudocomplementary peptide nucleic acids (“PNA”), a conventional sugar and internucleotide linkage has been replaced with a 2-aminoethylglycine amide backbone polymer (see, e.g., Nielsen et al., Science, 254:1497-1500, 1991; Egholm et al., J. Am. Chem. Soc., 114: 1895-1897 1992; Demidov et al., Proc. Natl. Acad. Sci. 99:5953-58, 2002; Peptide Nucleic Acids: Protocols and Applications, Nielsen, ed., Horizon Bioscience, 2004). The term “Tm enhancing nucleotide analog” as used herein refers to a nucleotide analog that, when incorporated into a primer or extension product, increases the annealing temperature of that primer or extension product relative to a primer or extension product with the same sequence comprising conventional nucleotides (A, C, G, and/or T), but not the Tm enhancing nucleotide analog. Those in the art will appreciate that Tm can be determined experimentally using well-known methods or can be estimated using algorithms, thus one can readily determine whether a particular nucleotide analog will serve as a Tm enhancing nucleotide analog when used in a particular context, without undue experimentation. A wide range of nucleotide analogs are available as triphosphates, phoshoramidites, or CPG derivatives for use in enzymatic incorporation or chemical synthesis from, among other sources, Glen Research, Sterling, Md.; Link Technologies, Lanarkshire, Scotland, UK; and TriLink BioTechnologies, San Diego, Calif. Descriptions of oligonucleotide synthesis and nucleotide analogs, can be found in, among other places, S. Verma and F. Eckstein, Ann. Rev. Biochem. 67:99-134 (1999); Goodchild, Bioconj. Chem. 1:165-87 (1990); Current Protocols in Nucleic Acid Chemistry, Beaucage et al., eds., John Wiley & Sons, 1999, including supplements through January 2005; and Nucleic Acids in Chemistry and Biology, 2d ed., Blackburn and Gait, eds., Oxford University Press, 1996.

### II. REAGENTS

The term “bisulfite treated gDNA” as used herein refers to gDNA that is sodium bisulfite treated, typically according to methods known on the art (see, e.g., Boyd and Zon, Anal. Biochem. 326:278-80, 2004; Frommer et al., Proc. Natl. Acad. Sci. 89:1827-31, 1992). During bisulfite treatment, cytosine residues are typically deaminated to uracil, while 5-methylcytosine (5mC) residues are generally non-reactive. When amplified, uracil residues are converted to thymines, while 5mC residues are amplified as cytosines. Thus, following bisulfite treatment, unmethylated sequences tend to be “U/T rich” compared to fully methylated sequences and the two DNA strands are no longer complementary. In the case of a mixed population sample, both a methylated “C rich” version and a “U/T rich” version of the “same” sequence are possible. Some cell populations may contain additional subpopulations with varying intermediate degrees of target cytosine methylation, resulting in yet additional counterparts of the “same” sequence with differing degrees of deaminated/converted cytosines. When mixed population bisulfite treated gDNA is amplified using conventional amplification methods, strand amplification bias typically occurs because, it is believed, the U/T rich strand is more efficiently amplified than the “C rich” highly methylated strand or other counterparts comprising intermediate levels of converted cytosine (see, e.g., Warnecke et al., Nucl. Acids Res. 25:4422-26, 1997). According to the disclosed methods, bisulfite treated gDNA is amplified under conditions designed to decrease strand amplification bias.

Within the bisulfite treated gDNA are regions of interest, referred to herein as “bisulfite treated gDNA target sequences” or “target sequences”, that are being evaluated to determine their methylation state (i.e., the presence, absence, or degree of methylation of one or more cytosines). Typically, such target sequences comprise a number of potential cytosine methylation sites, often within CpG islands. A potentially methylated cytosine within a target sequence whose methylation state is being evaluated is referred as a “target cytosine”. According to certain disclosed methods, a first primer pair is designed to anneal to sequences in the bisulfite treated gDNA that flank the target sequence so the target sequence can be amplified. Those in the art will appreciate that one primer of the first primer pair will comprise a sequence that is complementary or substantially complementary to a first flanking sequence in the bisulfite treated gDNA, while the corresponding primer of the primer of the primer pair comprises a sequence that is the same as or substantially the same as a second flanking sequence.

A “first primer pair” of the current teachings comprises a tailed first primer and a tailed second primer. The tailed first primer comprises (1) a target-complementary portion, and (2) a first primer-binding site that is located upstream of the target-complementary portion. The target-complementary portion of the first primer is designed to specifically hybridize under appropriate conditions with a region of the gDNA target sequence that is located downstream from the cytosine residue(s) whose methylation status is being evaluated. In some embodiments, all or at least a substantial part of the tailed first primer anneals with the complement of the tailed first primer in the second extension product, e.g., the complement of the target-complementary portion and the complement of the first primer-binding site. The tailed second primer of the first primer pair comprises (1) a first extension product-complementary portion that is designed to specifically hybridize with a complementary region of the first extension product and (2) a second primer-binding portion upstream of the first extension product-complementary portion. The first extension product-complementary portion of the second primer is typically the same as or substantially the same as a region of the gDNA target sequence that is located upstream from the cytosine residue(s) whose methylation status is being evaluated. In some embodiments, all or at least a substantial part of the tailed second primer anneals with the complement of the tailed first primer in the third extension product, e.g., the complement of the first extension product-complementary portion and the complement of the second primer-binding site.

In some embodiments, a nucleotide analog, for example but not limited to a Tm enhancing nucleotide analog, is incorporated into the target-complementary portion of the tailed first primer, the first extension product-complementary portion of the tailed second primer, or both. In some embodiments, a multiplicity of nucleotide analogs, including without limitation Tm enhancing nucleotide analogs, are incorporated into the target-complementary portion of the tailed first primer, the first extension product-complementary portion of the tailed second primer, or both. In some embodiments, the multiplicity of incorporated nucleotide analogs comprises the same nucleotide analog, while in other embodiments, the multiplicity of incorporated nucleotide analogs comprises at least two different nucleotide analogs. The primer-binding portions of the tails of the first primer and the second primer typically comprise all four natural nucleotides, while the corresponding target-complementary portion, the first extension product-complementary portion, or both, typically comprise three of the four natural nucleotides, due to the conversion of C to U/T, and can but need not comprise a nucleotide analog.

Certain disclosed methods further comprise a “second primer pair” that includes a third primer and a fourth primer. The third primer comprises a sequence that is complementary to, or at least substantially complementary to, the complement of the first primer-binding site incorporated in the second extension product. The fourth primer comprises a sequence that is complementary to, or at least substantially complementary to, the complement of the second primer-binding site incorporated in the first extension product. In some embodiments, the third primer, the fourth primer, or both, comprise an affinity tag, a reporter group, a reporter probe-binding site, a mobility modifier, or combinations thereof; in some embodiments, a third primer, a fourth primer, or both further comprise a tail, for example but not limited to a hybridization tag or a reporter probe-binding site. The third primer and the fourth primer typically comprise all four natural nucleotides (A, C, G, and T), allowing higher annealing temperatures to be employed relative to a counterpart sequence comprising three of the natural nucleotides but not the fourth. In some embodiments, the third primer, the fourth primer, or both, comprise the complement of a universal priming sequence.

A “universal priming sequence” is a generic sequence in a primer-binding site that is found in more than one species of extension product and to which a universal third primer or a universal fourth primer binds, provided that they comprise the complementary universal sequence. Thus, the same third primer species can be used to amplify at least two different second extension product species, the same fourth primer species can be used to amplify at least two different third extension product species, or the same third primer species and the same fourth primer species can be used to amplify at least two different second extension product species and at least two different third extension product species. Universal primers/priming sequences, including without limitation M13 universal primers and T7 universal primers, and their use are well known in the art. In some embodiments, a third primer, a fourth primer, or both, are used as sequencing primers for a subsequent detection/quantitation step, including without limitation, cycle sequencing, single nucleotide (base) extension sequencing, and solid phase sequencing; and either or both strands of a double-stranded molecule, for example, a second extension product:third extension product duplex, can be sequenced or otherwise detected (see, e.g., McPherson, particularly section 4 of Chapter 5).

The term “reporter probe” refers to a sequence of nucleotides, nucleotide analogs, or nucleotides and nucleotide analogs, that are designed to anneal with the reporter probe-binding site of a first extension product, a second extension product, a third extension product, an extension product surrogate, or combinations thereof, and when detected, including but not limited to a change in intensity or of emitted wavelength, is used to infer the presence, absence, and/or degree of methylation of the corresponding target cytosine(s). Most reporter probes can be categorized based on their mode of action, for example but not limited to: nuclease probes, including without limitation TaqMan® probes (see, e.g., Livak, Genetic Analysis: Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, or combinations thereof. In certain embodiments, reporter probes comprise a PNA, an LNA, or combinations thereof, and include stem-loop and stem-less reporter probe configurations. Certain reporter probes are singly-labeled, while other reporter probes are doubly-labeled. Dual probe systems that employ fluorescence resonance energy transfer (FRET) between adjacently hybridized probes are within the intended scope of the term reporter probe.

In certain embodiments, a reporter probe comprises a reporter group (including without limitation a fluorescent reporter group), a quencher (including without limitation dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TaqMan® probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Tyagi and Kramer, Nature Biotech. 14:303-08, 1995; Nazarenko et al., Nucl. Acids Res. 25:2516-21, 1997; Fiandaca et al., Genome Res. 11:609-13, 2001; Dubertret et al., Nature Biotech. 19:365-70, 2001; Zelphati et al., BioTechniques 28:304-15, 2000). In certain embodiments, reporter probes further comprise minor groove binders including but not limited to TaqMan® MGB probes and TaqMan® MGB-NFQ probes (both from Applied Biosystems). In certain embodiments, reporter probe detection comprises fluorescence polarization detection (see, e.g., Simeonov and Nikiforov, Nucl. Acids Res. 30:e91, 2002).

### III. TECHNIQUES

Amplification according to the present teachings encompasses any means by which at least a part of a bisulfite treated gDNA target sequence, a first extension product, a second extension product, a third extension product, surrogates thereof, or combinations thereof, is reproduced or copied, typically as a complementary strand in a template-dependent manner, including without limitation, linear or exponential amplification techniques. Exemplary means for performing an amplifying step include primer extension and PCR. Descriptions of certain amplification techniques can be found in, among other places, Sambrook and Russell, Molecular Cloning, A Laboratory Manual, Cold Spring Harbor Press, 3d ed., 2001 (“Sambrook and Russell”); Sambrook, Fritsch, and Maniatis, Molecular Cloning, A Laboratory Manual, Cold Spring Harbor Press, 2d ed., 1989 (“Sambrook et al.”); Current Protocols in Molecular Biology, Ausbel et al., eds. John Wiley & Sons, including supplements through January 2005 (“Ausbel”); PCR Primer: A Laboratory Manual, Diffenbach, Ed., Cold Spring Harbor Press (1995); and Rapley.

In certain embodiments, amplification comprises a cycle of the sequential steps of: (a) hybridizing a primer with complementary regions in a bisulfite treated gDNA target sequence, a first extension product, a second extension product, a third extension product, or combinations thereof; (b) synthesizing at least one strand of nucleotides in a template-dependent manner by extending the annealed primer using a polymerase; and (c) denaturing the newly-formed nucleic acid duplex to separate the strands. The cycle (a)-(c) may or may not be repeated. In some embodiments, steps (a) and (b) are performed at the same or nearly the same reaction temperature, in essence merging into one step. In certain embodiments, newly-formed nucleic acid duplexes may not be initially denatured, but can be used in their double-stranded form in one or more subsequent steps and either or both strands can, but need not, serve as surrogates of the target sequence. In certain embodiments, single-stranded extension products are generated and can, but need not, serve as target surrogates, for example but not limited to templates for a subsequent detection/quantitation step comprising sequencing.

Primer extension is an amplifying technique that comprises elongating a primer that is annealed to a template, for example a gDNA target sequence or an extension product, in the 5′=>3′ direction using an amplifying means such as a polymerase. According to certain embodiments, under appropriate conditions a polymerase can extend the annealed primer by incorporating nucleotides complementary to the template strand starting at the primer's 3′-end, to generate a complementary strand such as an extension product. In certain embodiments, the polymerase used for primer extension lacks or substantially lacks 5′-exonuclease activity.

In some embodiments, methods for reducing strand amplification bias comprise “touchdown PCR” or “hot start PCR” variations (see, e.g., McPherson, particularly sections 2.5 and 2.6 of Chapter 4). Certain methods for detecting amplified bisulfite treated gDNA target sequences comprise asymmetric PCR using a single primer or a primer pair where one of the primers is added in great excess compared to the other primer of that primer pair. Certain embodiments comprise multiplex amplification, for example but not limited to multiplex PCR. Those in the art will appreciate that multiplex amplification of bisulfite treated nucleic acid targets can be more difficult and tempermental than a single-plex reaction. Oftentimes, each target to be amplified in a multiplex reaction must first be optimized in a single-plex reaction. Typically multiplex amplification reactions require the optimization of reaction conditions, for example but not limited to, acceptable amplicon size, the concentrations of the various primers, including without limitation, tailed primer pairs and second primer pairs, and their annealing temperatures to arrive at an acceptable multiplex reaction condition. In some cases the lengths of the target-complementary portions or the primer-binding sites can be varied to change the Tm of that primer or primer pair. In some embodiments, a universal second primer pair can be used to simplify optimization. Such optimization, however, is routine and does not require undue experimentation (see, e.g., McPherson, particularly at section 10 of Chapter 10; and Rapley, particularly at Chapter 79).

Separating comprises any means for removing at least some unreacted components, at least some reagents, or both some unreacted components and some reagents from a first extension product, a second extension product, a third extension product, or combinations thereof, other than digestion. The skilled artisan will appreciate that a number of well-known separation means can be useful in the disclosed methods. Exemplary techniques for performing a separation step include gel electrophoresis, for example but not limited to, isoelectric focusing and capillary electrophoresis; dielectrophoresis; flow cytometry, including but not limited to fluorescence-activated sorting techniques using beads, microspheres, or the like; liquid chromatography, including without limitation, HPLC, FPLC, size exclusion (gel filtration) chromatography, affinity chromatography, ion exchange chromatography, hydrophobic interaction chromatography, immunoaffinity chromatography, and reverse phase chromatography; affinity tag binding; aptamer-target binding; hybridization tag-hybridization tag complement annealing; mass spectrometry, including without limitation MALDI-TOF, MALDI-TOF-TOF, ESI-TOF, tandem mass spec (MS-MS), LC-MS, and LC-MS/MS; a microfluidic device; and the like. In some embodiments, a separation technique is combined with or occurs immediately before a detection step. Discussion of separation techniques and separation-detection techniques, can be found in, among other places, Rapley; Sambrook et al.; Sambrook and Russell; Ausbel et al.; Capillary Electrophoresis: Theory and Practice, P. Grossman and J. Colburn, eds., Academic Press, 1992; The Expanding Role of Mass Spectrometry in Biotechnology, G. Siuzdak, MCC Press, 2003; PCT Publication No. WO 01/92579; and M. Ladisch, Bioseparations Engineering: Principles, Practice, and Economics, John Wiley & Sons, 2001.

The terms “detecting” and “detection” are used in a broad sense herein and encompass any technique by which presence, absence, and/or degree of target cytosine methylation is determined or inferred. In some embodiments, the presence of a surrogate is detected, directly or indirectly, allowing the presence, absence, and/or degree of target cytosine methylation to be inferred. For example but not limited to, detecting a family of labeled sequencing products obtained using an extension product template; or detecting the fluorescence generated when a nuclease reporter probe, annealed to an extension product, is cleaved by a polymerase, wherein the detectable signal or detectable change in signal serves as a surrogate for the corresponding extension product and thus the gDNA target sequence. In some embodiments, detecting further comprises quantitating the detectable signal, including without limitation, a real-time detection method, such as quantitative PCR (“Q-PCR”). In some embodiments, detecting comprises determining the sequence of a sequencing product or a family of sequencing products generated from an extension product template; in some embodiments, such detecting comprises quantitating a multiplicity of sequencing products.

In certain embodiments, a detecting step comprises an instrument, i.e., using an automated or semi-automated detecting means that can, but need not, comprise a computer algorithm. In certain embodiments, a detecting instrument comprises or is coupled to a device for graphically displaying the intensity of an observed or measured parameter of an extension product or its surrogate on a graph, monitor, electronic screen, magnetic media, scanner print-out, or other two- or three-dimensional display and/or recording the observed or measured parameter. In certain embodiments, the detecting step is combined with or is a continuation of at least one separating step, for example but not limited to a capillary electrophoresis instrument comprising at least one fluorescent scanner and at least one graphing, recording, or readout component; a chromatography column coupled with an absorbance monitor or fluorescence scanner and a graph recorder; a chromatography column coupled with a mass spectrometer comprising a recording and/or a detection component; or a microarray with a data recording device such as a scanner or CCD camera. In certain embodiments, the detecting step is combined with the amplifying step, for example but not limited to, real-time analysis such as Q-PCR. Exemplary means for performing a detecting step include the ABI PRISM® Genetic Analyzer instrument series, the ABI PRISM® DNA Analyzer instrument series, the ABI PRISM® Sequence Detection Systems instrument series, and the ABI PRISM® Real-Time PCR instrument series (all from Applied Biosystems); and microarrays and related software such as the Applied Biosystems microarray and Applied Biosystems 1700 Chemiluminescent Microarray Analyzer and other commercially available microarray and analysis systems available from Affymetrix, Agilent, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003) or bead array platforms (Illumina, San Diego, Calif.). Exemplary software includes GeneMapper™ Software, GeneScan® Analysis Software, and Genotyper® software (all from Applied Biosystems).

In certain embodiments, an extension product or an extension product surrogate does not comprise fluorescent reporter groups, but can be detected and quantified based on their corresponding mass-to-charge ratios (m/z). For example, in some embodiments, a primer (including without limitation, a tailed first primer, a tailed second primer, a third primer and/or a fourth primer) comprises a mass spectrometry-compatible reporter group, including without limitation, mass tags, charge tags, cleavable portions, or isotopes that are incorporated into first extension products, second extension products, third extension products, or their surrogates, and can be used for mass spectrometer detection (see, e.g., Haff and Smirnov, Nucl. Acids Res. 25:3749-50, 1997; and Sauer et al., Nucl. Acids Res. 31:e63, 2003). An extension product, a part of an extension product, or other extension product surrogate can be detected by mass spectrometry allowing the presence, absence, or degree of methylation of the corresponding target cytosine(s) to be inferred. In some embodiments, a primer comprises a restriction enzyme site, a cleavable portion, or the like, to facilitate release of a part of a subsequent extension product for detection. In certain embodiments, a multiplicity of surrogates, are separated by liquid chromatography or capillary electrophoresis, subjected to ESI or to MALDI, and detected by mass spectrometry. Descriptions of mass spectrometry can be found in, among other places, The Expanding Role of Mass Spectrometry in Biotechnology, Gary Siuzdak, MCC Press, 2003.

In certain embodiments, surrogates such as a reporter probe or a cleaved portion of a reporter probe, the reporter group of a released hybridization tag complement, or a part of a hybridization tag complement are detected, directly or indirectly. For example but not limited to, hybridizing an extension product to a labeled reporter probe comprising a quencher, including without limitation, a molecular beacon, including stem-loop and stem-free beacons, a TaqMan® probe, a LightSpeed™ PNA probe, or a microarray capture probe. In certain embodiments, the hybridization occurs in solution such as hybridizing a molecular beacon to first extension products, second extension products, third extension products, or their surrogates. In other embodiments, a first extension product, a second extension product, a third extension product, or a reporter probe is bound to a capture surface and upon hybridization of the corresponding reporter probe, first extension product, second extension product, or third extension product, and a detectable signal is emitted (see, e.g., EviArrays™ and EviProbes™, Evident Technologies).

In certain embodiments, detecting comprises measuring or quantifying the detectable signal of a reporter group or the change in a detectable signal of a reporter group, typically due to the presence of an extension product. For example but not limited to, an unhybridized reporter probe may emit a low level, but detectable signal that quantitatively increases when hybridized, including without limitation, certain molecular beacons, LNA probes, PNA probes, and light-up probes (see, e.g., Svanik et al., Analyt. Biochem. 281:26-35, 2000; Nikiforov and Jeong, Analyt. Biochem. 275:248-53, 1999; and Simeonov and Nikiforov, Nucl. Acids Res. 30:e91, 2002). In certain embodiments, detecting comprises measuring fluorescence polarization. Those in the art understand that the separation means and/or detecting means employed are generally not limiting. Rather, a wide variety of separation means and detecting means are within the scope of the disclosed methods and kits, provided that they allow the presence, absence, and/or degree of target cytosine(s) methylation to be inferred.

### IV. CERTAIN KITS

The instant teachings also provide kits designed to expedite performance of the subject methods. Kits serve to expedite the performance of the methods of interest by assembling two or more components required for carrying out the disclosed methods. Kits may contain components in pre-measured unit amounts to minimize the need for measurements by end-users. Kits may include instructions for performing one or more of the disclosed methods. Preferably, the kit components are optimized to operate in conjunction with one another.

In some embodiments, kits comprise a tailed first primer, a corresponding tailed second primer, a polymerase, and sodium bisulfite. Some kits comprise a multiplicity of different tailed primer pairs, a multiplicity of different second primer pairs, a nucleotide analog, or combinations thereof, to amplify and/or detect a multiplicity of different target sequences. Certain kits comprise a universal second primer pair. Certain kits further comprise a control target sequence, such as an “internal control” sequence or “standard” for assay calibration and/or validation purposes.

### V. EXEMPLARY EMBODIMENTS

The present teachings are generally directed to compositions, methods, and kits for amplifying bisulfite treated gDNA target sequences and for determining the methylation profile of those gDNA target sequences. Methods are disclosed for decreasing the strand amplification bias that occurs when amplifying bisulfite treated gDNA, particularly when such gDNA is obtained from a mixed population sample. Also disclosed are methods for detecting and for quantitating the methylation profile of target sequences using the extension products generated using the bias reducing amplification methods of the current teachings. The disclosed methods typically employ a tailed first primer pair that can, but need not, comprise nucleotide analogs that alter the Tm of the primers relative to comparable primers consisting of A, C, G, and T, but not nucleotide analogs.

The gDNA is typically sodium bisulfite treated, according to methods known on the art (see, e.g., Boyd and Zon, Anal. Biochem. 326:278-80, 2004). During bisulfite treatment, cytosine residues are typically deaminated to uracil, while 5-methylcytosine (5mC) residues are generally non-reactive. When amplified, uracil residues are converted to thymines, while 5mC residues are amplified as cytosines. For illustration purposes, a hypothetical target sequence consisting of 25% each of A, C, G, and T, if unmethylated would be converted to 25% A, 25% G, and 50% T by bisulfite treatment; the same hypothetical target sequence, if fully methylated, would retain the original composition of 25% A, 25% C (5mC), 25% G, and 25% T. Thus, the methylated sequence would have a higher (G+C) content than the unmethylated counterpart of the “same” sequence. As a consequence of their higher (G+C) content, methylated duplex target sequences will have higher melting temperatures than their unmethylated counterparts and their denatured strands will have more secondary structure than their unmethylated single-stranded counterparts (Warnecke et al., Nucl. Acids Res. 25:4422, 1997; Voss et al., Anal. Chem. 70:3818, 1998). The increased melting temperature and increased secondary structure are believed to explain, at least in part, the strand amplification bias (also referred to as “PCR bias”) in favor of the unmethylated target sequences (Id.).

To overcome increased duplex melting temperatures and secondary structure associated with the bias against methylated strand amplification, amplification may be performed at higher temperatures. However, this presents additional problems with respect to primer design. For example, to achieve a Tm of 60° C., the sequence-complementary portions of primers used for amplifying bisulfite-treated gDNA are often 20-25 nucleotides long or even longer. These longer gDNA primers increase the possibility of partial sequence mismatch at the 3′-end, resulting in non-specific amplification. Additionally, the high percentage of T residues and/or A residues in converted DNA and its complement makes designing primers with good selectivity difficult and increase the possibility of primer dimer formation as well as other amplification artifacts (see, e.g., Tusnady et al, Nucl. Acids Res. 33:e9, 2005).

Certain embodiments of the current teachings allow increased amplification reaction temperatures to be used while decreasing PCR bias and certain other amplification artifacts. In some embodiments, the tailed first primer pairs comprise a first primer and/or a second primer that include at least one Tm enhancing nucleotide analog in their respective target-complementary portion or first extension product-complementary portion that increase the Tm of the subject primers relative to the same primer consisting of A, C, G, and T. In some embodiments, at least one Tm enhancing nucleotide analog is incorporated into a first extension product, a second extension product, a third extension product, or combinations thereof, during primer extension. Those in the art will appreciate that any number of nucleotide analogs can be selectively incorporated into primers during oligonucleotide synthesis using, for example but not limited to, phosphoramidite chemistry techniques known in the art and automated DNA synthesizers. Exemplary Tm enhancing nucleotide analogs for synthetic incorporation include C-5 propynyl-dc or 5-methyl-2′-deoxycytidine substituted for dC; 2,6-diaminopurine 2′-deoxyriboside (2-amino-dA) substituted for dA; and C-5 propynyl-dU for dT; which increase the relative melting temperature approximately 2.8° C., 1.3° C., 3.0° C., and 1.7° C. per substitution, respectively. In certain embodiments of the disclosed methods, higher annealing and/or extension temperatures are possible because tailed first primers and/or tailed second primers comprising Tm enhancing analogs are used.

The first and the second primers of the current teachings also comprise upstream tails that include primer-binding sites for annealing third and fourth primers. These primer-binding sites and the third and fourth primers can include all four of the conventional nucleotides, increasing their specificity compared to the bisulfite-converted gDNA or first extension product sequences to which the target-complementary portion of the tailed first primer or the first extension product-complementary portion of the tailed second primer anneal. The annealing temperatures of the third and fourth primers can also be higher due to higher G:C content. After the first round of amplification, the first extension product and the second extension product comprise the tailed portion or the complement of the tailed portion to which the third and fourth primers can anneal and can drive subsequent rounds of amplification at higher reaction temperatures relative to certain conventional bisulfite-treated gDNA primers. In certain embodiments, a third primer, a fourth primer, or both comprise a universal primer sequence so the same third primer species can be used to amplify at least two different second extension products and/or the same fourth primer species can be used to amplify at least two different third extension products.

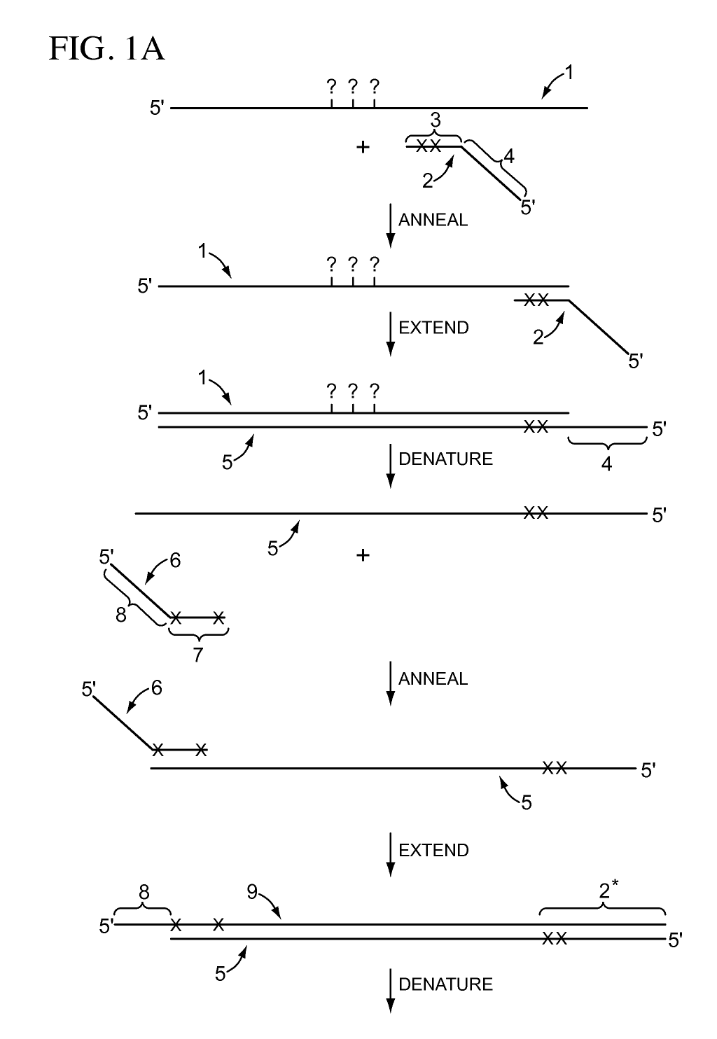

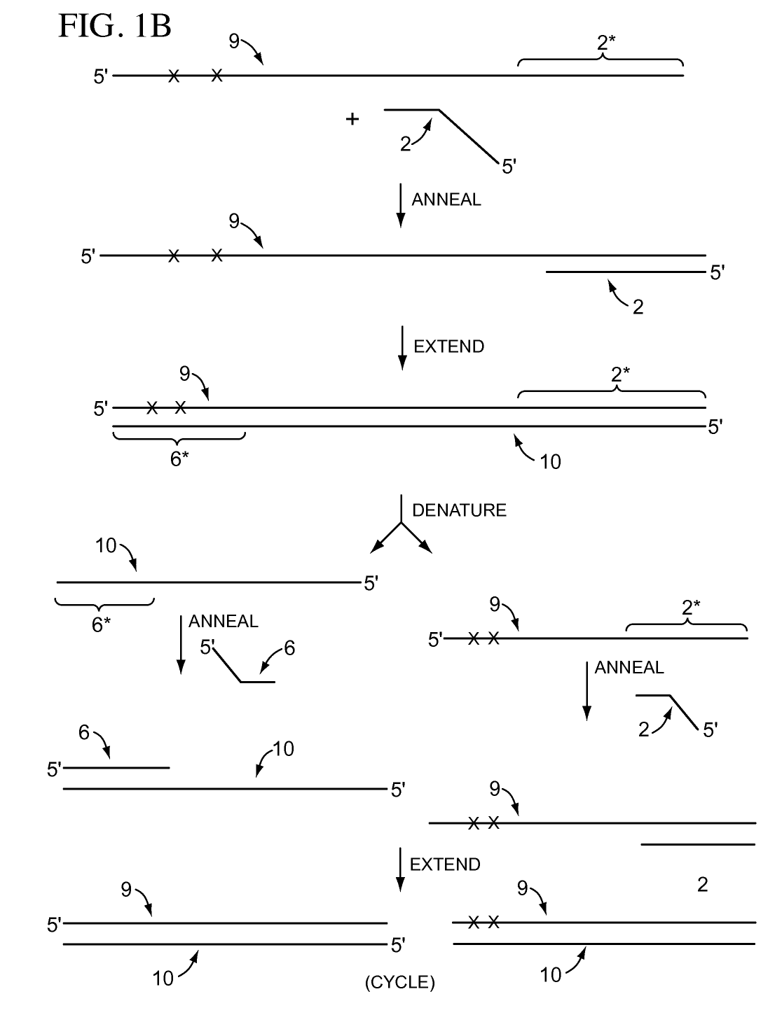

In one illustrative embodiment, shown in FIGS. 1A and 1B, a bisulfite treated gDNA sequence 1 is combined with a corresponding tailed first primer 2 comprising a target-complementary portion 3 and an upstream tail 4 comprising a first primer-binding site that typically includes A, C, G, and T. In this example, the target-complementary portion further comprises two Tm enhancing nucleotide analogs (shown by “X”). Under suitable conditions, the gDNA 1 anneals with target-complementary portion 3 of the tailed first primer 2, and the annealed first primer is extended to generate a first extension product 5. The duplex comprising the first extension product 5 and the gDNA 1 is denatured, releasing the first extension product 5. A tailed second primer 6, comprising a first extension product-complementary portion 7 that includes two Tm enhancing nucleotide analogs (shown by “X”) and upstream, a second primer binding site 8, anneals with the first extension product 5 via the first extension product-complementary portion 7. The annealed second primer 6 is extended to generate a second extension product 9 that comprises the complement of the incorporated first primer (shown as “2*”) on its 3′-end and the second primer binding site 8 on the 5′-end. The resulting duplex is denatured, releasing the second extension product 9 and the first extension product 5. Under suitable conditions, another tailed first primer 2 anneals to the complement of the incorporated first primer 2* in the second extension product 9, and is extended to generate a third extension product 10, comprising the complement of the incorporated tailed second primer (shown as “6*”). The duplex comprising the third extension product 10 and the second extension product 9 is denatured. Another second primer 6 anneals with the complement of the incorporated second primer 6* in the third extension product 10 and is extended to generate an additional second extension product 9. Another first primer 2 anneals with the complement of the incorporated first primer 2* in the second extension product 9 and is extended to generate an additional third extension product 10. In some embodiments, the steps of annealing first primers 2 with second extension products 9, annealing second primers 6 with third extension products 10; extending the annealed primers to generate additional third extension products and additional second extension products; and denaturing the resulting duplexes are repeated (cycled) one or more times to generate a multiplicity of second and third extension products, typically by geometric amplification such as PCR. In some embodiments, the annealing temperature and the extending temperature are the same or essentially the same and thus, the two steps can be combined. It will be appreciated that for purposes of this application and the appended claims, such cycling between the annealing-extension temperature and the denaturation temperature is to be considered within the scope of three step cycling, i.e., annealing, extending, and denaturing, even though only two temperatures are used, annealing/extending and denaturing.

In some embodiments, a first primer does not contain a nucleotide analog; in some embodiments, a second primer does not contain a nucleotide analog. In some embodiments, a nucleotide analog is enzymatically incorporated into an extension product during primer extension. In some embodiments, a first primer, a second primer, a third primer, a fourth primer, or combinations thereof, comprises a reporter group, a hybridization tag, an affinity tag, a mobility modifier, a reporter probe-binding site, or combinations thereof. In some embodiments, a first extension product, a second extension product, a third extension product, or combinations thereof, comprises a reporter group, a hybridization tag, an affinity tag, a mobility modifier, a reporter probe-binding site, or combinations thereof.

According to certain embodiments of the disclosed methods, a tailed first primer species is used to amplify a bisulfite treated gDNA sequence and also to amplify a second extension product and a tailed second primer species is used to amplify a first extension product and also to amplify a third extension product (see, e.g., FIGS. 1A and 1B). In some embodiments, a third primer is used to amplify a second extension product and/or a fourth primer is used to amplify a third extension product. In some embodiments, one third primer species is used to amplify a multiplicity of different species of second extension product, one fourth primer species is used to amplify a multiplicity of different species of third extension product, or one third primer species is used to amplify a multiplicity of different species of second extension product and one fourth primer species is used to amplify a multiplicity of different species of third extension product.

According to certain methods, including without limitation, embodiments wherein the first primer pair does not comprise Tm enhancing nucleotide analogs, two different thermocycling profiles are employed in a two-stage amplification technique to decrease strand amplification bias. For example, the first stage comprises a limited number of extension steps, typically at least two (generating a first extension product and a second extension product), and is generally performed using a lower temperature thermocycling profile. The target-complementary portion of the tailed first primer, and typically only the target-complementary portion, anneals with the corresponding region of the bisulfite treated gDNA and a first extension product comprising the tail portion of the first primer is generated. The first extension product-complementary portion of the tailed second primer, and typically only the first extension product-complementary portion, anneals with the corresponding region of the first extension product and a second extension product comprising the tail portion of the second primer is generated. After two or a few extension steps, the reaction is shifted to a higher temperature thermocycling profile. In the second stage of the amplification reaction, additional tailed first primers anneal with the second extension product to generate third extension products and additional tailed second primers anneal with the third extension products to generate additional second extension products. Higher temperatures can be used in this second amplification stage since both the target-complementary portion and the first primer-binding portion of the tailed first primers participate in the annealing with the second extension product. Likewise, both the first extension product-complementary portion and the second primer-binding portion of tailed second primers participate in the annealing with the third extension product. In contrast, only the target-complementary portion of the tailed first primer anneals with the bisulfite treated gDNA and only the first extension product-complementary portion of the tailed second primer anneals with the first extension product since neither of these templates comprise a corresponding primer-binding portion.

Alternatively, a second primer pair is used at the higher thermocycling profile to generate second extension products and third extension products in the second stage. In these embodiments, higher temperature thermocycling profiles are possible since the third and fourth primers typically comprise all four of the conventional nucleotides and can form more G:C pairs than when a bisulfite converted, and therefore C-deficient, sequence is used for annealing. Additionally, the lengths of the primer-binding portions can be designed with a length and G:C content necessary for higher annealing temperatures. In contrast, the binding regions of the gDNA are often size limited because CpG motifs are typically avoided and after conversion, the gDNA binding regions are C-deficient, decreasing possible G:C pairing between the template and the primer.

Thus, in some embodiments, a tailed first primer anneals with the bisulfite treated gDNA (typically via its target-complementary portion) at a first annealing temperature and a tailed second primer anneals with the first extension product (typically via its first extension product-complementary portion) at a second annealing temperature. Typically, the first annealing temperature and the second annealing temperature are the same or substantially the same (e.g., ±3° C.). In some embodiments, another tailed first primer anneals with a second extension product at a third annealing temperature, wherein both the target-complementary portion and the first primer-binding portion of the tailed first primer anneal with the second extension product. Another tailed second primer anneals with a third extension product at a fourth annealing temperature, wherein both the first extension product-complementary portion and the second primer-binding portion of the tailed second primer anneal with the third extension product. In some embodiments, the third and fourth annealing temperatures are the same or substantially the same (e.g., ±3° C.), but the third annealing temperature is at least 5° C. higher than the first annealing temperature, and in some embodiments, at least 10° C. higher than the first annealing temperature. In other embodiments, the first annealing temperature, the second annealing temperature, the third annealing temperature, and the fourth annealing temperature are the same or substantially the same.

In other embodiments, a third primer anneals with the second extension product at a third annealing temperature and a fourth primer anneals with the third extension product at a fourth annealing temperature. In certain of these embodiments, the third and fourth annealing temperatures are the same or substantially the same (e.g., +3° C.), but the third annealing temperature is at least 5° C. higher than the first annealing temperature, and in some embodiments, at least 10° C. higher than the first annealing temperature. In some embodiments, the first annealing temperature, the second annealing temperature, the third annealing temperature, and the fourth annealing temperature are the same or substantially the same. In some embodiments of the disclosed methods, the annealing temperatures of a thermocycling profile are sufficiently high that the annealing steps and the extension steps can be performed at the same temperature or substantially the same temperature.

Certain disclosed methods comprise a step of detecting an extension product of the disclosed methods or the surrogate of such an extension product, and the methylation state of the corresponding target cytosine is inferred. Thus, in some methods, a first extension product, a second extension product, a third extension product, a surrogate of an extension product, or combinations thereof are detected. Exemplary detecting means include without limitation mobility dependent analytical techniques, such as capillary electrophoresis; microarray detection (including fixed and bead arrays); and mass spectrometry. In some embodiments, an extension product or its surrogate comprises a reporter group that is detected, for example but not limited to, a labeled third extension product or a ZipChute® comprising a fluorescent reporter group. In some embodiments, unlabeled or indirectly labeled extension products are detected, for example but not limited to end point detection using reporter probes, including without limitation molecular beacons or nuclease probes, such as a TaqMan™ probe, that serve as extension product surrogates.

In some embodiments, detecting further comprises quantitating an extension product or its surrogate, including without limitation Q-PCR or sequencing. In some embodiments, quantitating includes the use of internal standards, control sequences, standard curves or calibration curves (see, e.g., Voss et al., Anal. Biochem. 70:3818-23, 1998). In some embodiments, detecting comprises determining the identity of or the quantity of a single-stranded extension product, including without limitation, a first extension product, a second extension product, a third extension product, a surrogate of a single-stranded extension product, or combinations thereof, generated by any means, including but not limited to denaturation of a duplex comprising an extension product, a single-stranded amplicon generated by asymmetric PCR, or the like. In some embodiments, detecting comprises determining the identity of or the quantity of a double-stranded molecule, for example but not limited to, a duplex comprising at least one extension product, such as a first extension product:second extension product duplex.

Although many of the embodiments of the present teachings have been discussed largely in the context of methylation analysis of bisulfite treated samples, it will be appreciated that the present teachings can be applied in other contexts, and more generally allow for the reduction of bias associated with amplifying two versions of a nucleic acid sequence that share the same (or roughly the same) primer sites, but differ in the base composition of the amplicon internal to the primer sites. As a hypothetical example, the present teachings contemplate a scenario in which a genomic locus A is under investigation between a first pool of samples (pool 1) and a second pool of samples (pool 2). For example, pool 1 can be a collection of pooled samples from normal patients, and pool 2 can be a collection of pooled samples from diseased patients. In such a scenario, an experimentalist might wish to concurrently PCR amplify locus A in pool 1 and pool 2, and quantify the nucleotide differences (for example SNPs) between pool 1 and pool 2, using for example SNP-specific probes. If the genomic locus A in pool 1 comprises A:T rich sequences, and the genomic locus A in pool 2 comprises G:C rich sequences, concurrent amplification of the samples using conventional methods would result in preferential amplification of the A:T rich pool 1, thus precluding accurate quantitation of differences in locus A between pool 1 and pool 2. However, the present teachings can minimize this bias in amplifying the two related sequences through the presence of nucleotide analogs, tailed primers, and other approaches as provided herein.

Aspects of the present teachings may be further understood in light of the following examples. These examples are intended for illustration purposes only, and should not be construed as limiting the scope of the present teachings in any way.

### Example 1

A first primer pair, designed to anneal with a bisulfite treated RasSF target sequence in gDNA, is synthesized using phosphoramidite chemistry and an automated DNA synthesizer according to the manufacturer's instructions. The illustrative tailed first primer comprises the sequence: CAGGAAACAGCTATGACCCTA*CA*CCCA*A*A*TTTCCA*TTA* (SEQ ID NO:1), including a first primer-binding site comprising a universal M13 primer sequence (shown in italics) upstream from the target-complementary portion (shown underlined). The target-complementary portion comprises the nucleotide analog 2-amino-dA (shown as “A*”). The illustrative tailed second primer comprises the sequence: TGTAAAACGACGGCCAGTTA*GTTTA*A*TGA*GTTTA*GGTTTTTT (SEQ ID NO:2), including a second primer-binding site comprising a different universal M13 primer sequence (shown in italics) upstream from the first extension product-complementary portion (shown underlined). The first extension product-complementary portion also comprises the nucleotide analog 2-amino-dA (shown as “A*”). The calculated Tm for the target-complementary portion of the first primer is 52° C. when no nucleotide analogs are incorporated, but increases to 73° C. when 2-amino-dA is incorporated, as shown. The calculated Tm for first extension product-complementary portion of the second primer is 53° C. when no nucleotide analogs are incorporated, but increases to 68° C. when 2-amino-dA is incorporated, as shown. (2-amino-dA is commercially available as a phosphoramidite for use with automated DNA synthesizers from, among other sources, Glen Research, Sterling Va.; catalog #10-1085-xx).

The primer pair (0.25 μL first primer, 0.25 μL second primer, 5 μM each) is combined with 0.5 μL bisulfite treated gDNA (10 ng/μL), 1 μL AmpliTaq Gold 10× buffer (Applied Biosystems, Foster City, Calif.), 0.8 μL dNTPs (2.5 mM each), 0.8 μL MgCl₂ (25 nM), 0.2 μL AmpliTaq Gold polymerase (Applied Biosystems) and 6.2 μL water in a 0.2 mL MicroAmp® sample tube (N8010580, Applied Biosystems) to form a reaction composition. The tube is capped with a MicroAmp® Tube Cap (N8010534, Applied Biosystems), placed in a MicroAmp® 96-well tray retainer (P/N 403081), and the tray is placed in a thermocycler and heated to 95° C. for 11 minutes to activate the polymerase. The reaction composition is cycled thirty-five times at 97° C. for 5 seconds, 70° C. for 2 minutes, and 72° C. for 45 seconds, then cooled to 4° C.

### Example 2

To compare the sequencing results obtained for a region of a bisulfite treated gDNA BrcA target sequence, an untailed first primer pair and a corresponding tailed first primer pair were synthesized. The untailed first primer pair included a first primer with the sequence: AACAAACTAAATAACCAATCCAAAAC (SEQ ID NO:3) and a second primer with the sequence TTAGAGTAGAGGGTGAAGGTTTTTT (SEQ ID NO:4). The corresponding tailed first primer pair included a first primer with the sequence: CAGGAAACAGCTATGACCAACAAACTAAATAACCAATCCAAAAC (SEQ ID NO:5), including a first primer-binding site comprising a universal M13 primer sequence (shown in italics) upstream from the target-complementary portion (shown underlined); and a second primer with the sequence: TGTAAAACGACGGCCAGTTTAGAGTAGAGGGTGAAGGTTTTTT (SEQ ID NO:6), including a second primer-binding site comprising a different universal M13 primer sequence (shown in italics) upstream from the first extension product-complementary portion (shown underlined).

Two reaction compositions, each comprising 10 μL total volume, including 1 μL AmpliTaq Gold 10× buffer, 0.8 μL dNTPs (2.5 mM each), 0.8 μL MgCl₂ (25 nM), 0.2 μL AmpliTaq Gold polymerase, 0.5 μL bisulfite treated gDNA (10 ng/μL), 6.2 μL water, and either (i) 0.25 μL untailed first primer (5 μM) and 0.25 μL untailed second primer (5 μM) or (ii) 0.25 μL tailed first primer (5 μM) and 0.25 μL tailed second primer (5 μM), were formed in capped MicroAmp® tubes, as described in Example 1, then the tray was placed in a thermocycler. The two reaction compositions were heated in parallel to 95° C. for 11 minutes to activate the polymerase, then cycled forty times in parallel at 95° C. for 30 seconds, 67° C. for 45 seconds, and 72° C. for 2 minutes, then cooled to 4° C.

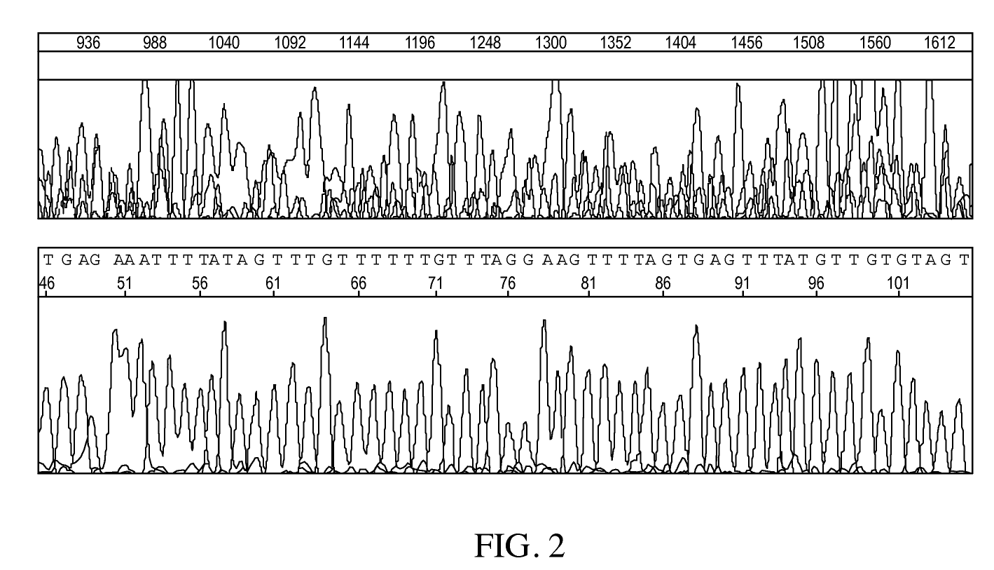

To remove unincorporated dNTPs and single-stranded primers, 1 μL Exo SAP-IT® reagent (#78201, USB Corporation, Cleveland, Ohio) was added per 10 μL cycled reaction composition. The tray was heated to 37° C. for 30 minutes, 80° C. for 15 minutes, then cooled to 4° C. For sequencing, 8 μL Big Dye Terminator v1.1 Ready Reaction Mix (Applied Biosystems), 1 μL sequencing primer (3.2 μM of the −21 M13 primers CAGGAAACAGCTATGACC (SEQ ID NO:7) or TGTAAAACGACGGCCAGT (SEQ ID NO:8), 10 μL water and 1 μL of one of the two treated cycled reaction composition were combined in separate tubes, as described in Example 1 and the tray with the two sequencing compositions was heated at 96° C. for 1 minute, then cycled 25 times at 96° C. for 10 seconds and 50° C. for four minutes, then cooled to 4° C. The two sequencing compositions were evaluated using an ABI PRISM® 3730 DNA Analyzer (Applied Biosystems). As shown in FIG. 2, no discernible sequence was obtained using the untailed first primer pair (top panel), in contrast to the sequence results obtained using the corresponding tailed first primer pair (bottom panel). The methylation status of individual cytosines in the gDNA sample can be inferred by comparing the sequence obtained from the extension products with the consensus sequence and identifying which Cs were converted to T as the result of bisulfite treatment and which were not converted.

### Example 3

To demonstrate the effect of nucleotide analog incorporation during primer extension, a tailed first primer pair was designed to amplify a region of bisulfite treated gDNA comprising a RasSF target sequence. The tailed first primer comprised the sequence: CAGGAAACAGCTATGACCCTACACCCAAATTTCCATTA (SEQ ID NO:9), including a first primer-binding site comprising a universal M13 primer sequence (shown in italics) upstream from the target-complementary portion (shown underlined); and a tailed second primer with the sequence: TGTAAAACGACGGCCAGTTAGTTTAATGAGTTTAGGTTTTTT (SEQ ID NO:10), including a second primer-binding site comprising a different universal M13 primer sequence (shown in italics) upstream from the first extension product-complementary portion (shown underlined). This tailed first primer pair was used to amplify the RasSF target in (i) a reaction composition comprising dCTP, but not dMeCTP, or (ii) a reaction composition comprising dMeCTP, but not dCTP. Two reaction compositions were formed, each comprising 1 μL AmpliTaq Gold 10× buffer, 0.8 μL MgCl₂ (25 mM), 0.2 μL AmpliTaq Gold polymerase, 0.25 μL tailed first primer (5 μM), 0.25 μL tailed second primer (5 μM), 0.5 μL bisulfite treated gDNA, and 0.8 μL dNTP mixture comprising dATP, dGTP, dTTP, and either dCTP or 5-methyl-dCTP (2.5 mM each). The two reaction mixtures were heated in parallel to 95° C. for 11 minutes, cycled forty times at 95° C. for 30 seconds, 67° C. for 45 seconds, and 72° C. for 2 minutes, then cooled to 4° C. The cycled reaction compositions were treated with Exo SAP-IT®, then sequenced as described in Example 2.

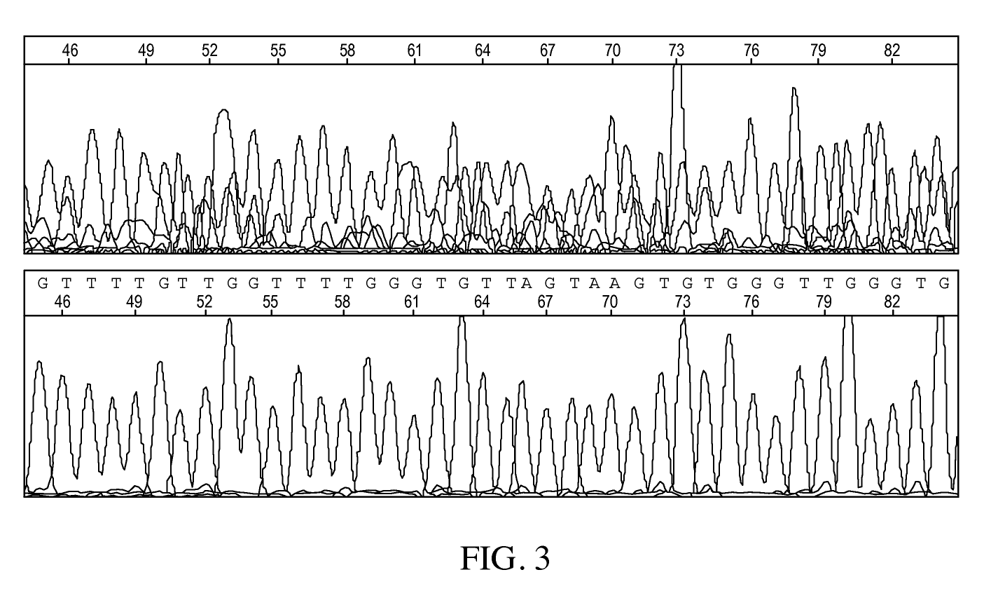

As shown in FIG. 3, in this example better sequence information was obtained, and therefore better methylation analysis is possible, when bisulfite treated samples were amplified under conditions where the nucleotide analog 5mC was incorporated in the absence of C (bottom panel) than vice versa (top panel).

### Example 4

Human gDNA is bisulfite treated using a published protocol (Boyd and Zon, Anal. Biochem. 326: 278-280, 2004; see also U.S. Provisional Patent Application Ser. Nos. 60/499,113; 60/520,942; 60/499,106; 60/523,054; 60/498,996; 60/520,941; 60/499,082; and 60/523,056). A tailed first primer pair is synthesized; the sequences of the tailed first primer and tailed second primer are: CAGGAAACAGCTATGACC[CTACACCCAAATTTCCATTA] (SEQ ID NO:11) and TGTAAAACGACGGCCAGTTAGTTTAATGAGTTTAGGTTTTTT (SEQ ID NO:12), respectively. The target-complementary portion of the tailed first primer is shown in brackets; the first extension product-complementary portion of the tailed second primer is shown in italics; and the respective tails, each comprising a primer-binding site, are shown underlined. In this exemplary tailed primer pair, the primer-binding sites comprise M13 sequences.

The bisulfite-treated gDNA nucleic acid target being interrogated comprises the sequence: TAGTTTAATGAGTTTAGGTTTTTTCGATATGGTTCGGTTGGGTTCGTGTTTCGTTGGT TTTGGGCGTTAGTAAGCGCGGGTCGGGCGGGGTTATAGGGCGGGTTTCGATTTTAG CGTTTTTTTTAGGATTTAGATTGGGCGGCGGGAAGGAGTTGAGGAGAGTCGCG[TAA TGGAAATTTGGGTGTAG] (SEQ ID NO:13), wherein the first region (to which the target-complementary portion of the tailed first primer anneals) is shown in brackets, the second region (the complement of which anneals with the first extension product-complementary portion of the tailed second primer) is shown in italics, and the potentially methylated target cytosines are shown underlined.

A reaction composition comprising 1 μL AmpliTaq Gold 10× buffer, 0.8 μL dNTPs (2.5 mM each), 0.8 μL MgCl₂ (25 nM), 0.2 μL AmpliTaq Gold polymerase, 0.5 μL bisulfite treated gDNA (10 ng/μL), 6.2 μL water, 0.25 μL (5 μM) of the tailed first primer (5 μM) and 0.25 μL (5 μM) tailed second primer is formed in a capped MicroAmp® tube, as described in Example 1, and the tray is placed in a thermocycler. The reaction composition is heated to 95° C. for 11 minutes to activate the polymerase, then cycled thirty-five times between 97° C. for 5 seconds, 60° C. for 2 seconds (typically 5-10° C. higher than the calculated Tm of the respective complementary portions of the tailed primer pair), and 72° C. for 45 seconds, then cooled to 4° C. The amplified reaction composition is treated with Exo SAP-IT® reagent, as described in Example 2. The digested amplified reaction composition can be subjected to a variety of further analyses, for example but not limited to sequencing, and the methylation status of the respective target cytosines inferred, with or without further amplification using additional tailed first primers and additional tailed second primers and/or the corresponding second primer pair.

Those in the art will understand that the compositions and methods of the current teachings can be applied, with appropriate target-specific modifications, to detect and/or quantify the methylation state of any number of bisulfite treated gDNA targets.

Although the disclosed teachings have been described with reference to various applications, methods, and compositions, it will be appreciated that various changes and modifications may be made without departing from the teachings herein. The foregoing examples are provided to better illustrate the disclosed teachings and are not intended to limit the scope of the current teachings in any way.

What is claimed is:

1. A method for reducing strand amplification bias with bisulfite treated genomic DNA (gDNA) comprising:
(a) annealing a first primer with the bisulfite treated gDNA at a first annealing temperature, wherein the first primer comprises: (i) a target-complementary portion and (ii) a first primer-binding site upstream from the target-complementary portion;
(b) extending the annealed first primer to generate a first extension product;
(c) annealing a second primer with the first extension product at a second annealing temperature, wherein the second primer comprises: (i) a first extension product-complementary portion and (ii) a second primer-binding site upstream from the first extension product-complementary portion;
(d) extending the annealed second primer to generate a second extension product;
(e) annealing an additional first primer with the second extension product at a third annealing temperature;
(f) extending the annealed first primer to generate a third extension product;
(g) annealing an additional second primer with the third extension product at a fourth annealing temperature;
(h) extending the annealed second primer to generate an additional second extension product; and
(i) optionally, repeating steps (e)-(h) at least one additional cycle.

2. The method of claim 1, wherein the first annealing temperature, the second annealing temperature, the third annealing temperature, and the fourth annealing temperature are the same or substantially the same.

3. The method of claim 1, wherein the first annealing temperature is at least five degrees Celsius (° C.) less than the third annealing temperature.

4. The method of claim 3, wherein the first annealing temperature is at least ten ° C. less than the third annealing temperature

5. The method of claim 3, wherein (i) the first annealing temperature and the second annealing temperature are the same or substantially the same and (ii) the third annealing temperature and the fourth annealing temperature are the same or substantially the same.

6. The method of claim 1, further comprising detecting the first extension product, the second extension product, the third extension product, a surrogate of an extension product, or combinations thereof.

7. The method of claim 6, wherein the detecting comprises quantitating the first extension product, the second extension product, the third extension product, the surrogate of an extension product, or combinations thereof.

8. A method for reducing strand amplification bias with bisulfite treated gDNA comprising:
(a) annealing a first primer with the bisulfite treated gDNA at a first annealing temperature, wherein the first primer comprises: (i) a target-complementary portion comprising a nucleotide analog and (ii) a first primer-binding site upstream from the target-complementary portion;
(b) extending the annealed first primer to generate a first extension product;
(c) annealing a second primer with the first extension product at a second annealing temperature, wherein the second primer comprises: (i) a first extension product-complementary portion comprising a nucleotide analog and (ii) a second primer-binding site upstream from the first extension product-complementary portion;
(d) extending the annealed second primer to generate a second extension product;
(e) annealing an additional first primer with the second extension product at a third annealing temperature;
(f) extending the annealed first primer to generate a third extension product;
(g) annealing an additional second primer with the third extension product at a fourth annealing temperature;
(h) extending the annealed second primer to generate an additional second extension product; and
(i) optionally, repeating steps (e)-(h) at least one additional cycle.

9. The method of claim 8, wherein the first annealing temperature, the second annealing temperature, the third annealing temperature, and the fourth annealing temperature are the same or substantially the same.

10. The method of claim 8, wherein the first annealing temperature is at least five ° C. less than the third annealing temperature.

11. The method of claim 10, wherein the first annealing temperature is at least ten ° C. less than the third annealing temperature

12. The method of claim 10, wherein (i) the first annealing temperature and the second annealing temperature are the same or substantially the same and (ii) the third annealing temperature and the fourth annealing temperature are the same or substantially the same.

13. The method of claim 8, wherein the target-complementary portion comprises a multiplicity of nucleotide analogs, the first extension product-complementary portion comprises a multiplicity of nucleotide analogs, or the target-complementary portion and the first extension product-complementary portion each comprise a multiplicity of nucleotide analogs.

14. The method of claim 8, wherein the nucleotide analog comprises a 5-methylcytosine, a 2-amino adenine (2-amino-dA), a C-5 propynyl-dc, a C-5 propynyl-dU, a locked nucleic acid (LNA), a 2′-O-methyl nucleotide, a phosphoroamidate nucleotide, or combinations thereof.

15. The method of claim 8, further comprising detecting the first extension product, the second extension product, the third extension product, or combinations thereof.

16. The method of claim 15, wherein the detecting comprises quantitating the first extension product, the second extension product, the third extension product, or combinations thereof.

17. A method for reducing strand amplification bias with bisulfite treated gDNA comprising:
(a) annealing a first primer with the bisulfite treated gDNA at a first annealing temperature, wherein the first primer comprises: (i) a target-complementary portion and (ii) a first primer-binding site upstream from the target-complementary portion;
(b) extending the annealed first primer to generate a first extension product comprising a nucleotide analog;
(c) annealing a second primer with the first extension product at a second annealing temperature, wherein the second primer comprises: (i) a first extension product-complementary portion and (ii) a second primer-binding site upstream from the first extension product-complementary portion;
(d) extending the annealed second primer to generate a second extension product comprising a nucleotide analog;
(e) annealing an additional first primer with the second extension product at a third annealing temperature;
(f) extending the annealed first primer to generate a third extension product;
(g) annealing an additional second primer with the third extension product at a fourth annealing temperature;
(h) extending the annealed second primer to generate an additional second extension product; and
(i) optionally, repeating steps (e)-(h) at least one additional cycle.

18. The method of claim 17, wherein the first annealing temperature, the second annealing temperature, the third annealing temperature, and the fourth annealing temperature are the same or substantially the same.

19. The method of claim 17, wherein the first annealing temperature is at least five ° C. less than the third annealing temperature.

20. The method of claim 19, wherein the first annealing temperature is at least ten ° C. less than the third annealing temperature

21. The method of claim 19, wherein (i) the first annealing temperature and the second annealing temperature are the same or substantially the same and (ii) the third annealing temperature and the fourth annealing temperature are the same or substantially the same.

22. The method of claim 17, wherein the first extension product comprises a multiplicity of nucleotide analogs, the second extension product comprises a multiplicity of nucleotide analogs, or the first extension product and the second extension product each comprise a multiplicity of nucleotide analogs.

23. The method of claim 17, wherein the nucleotide analog comprises a 5-methylcytosine, a 2-amino-dA, a C-5 propynyl-dc, a C-5 propynyl-dU, a LNA, a 2′-O-methyl nucleotide, a phosphoroamidate nucleotide, or combinations thereof.+

24. The method of claim 17, further comprising detecting the first extension product, the second extension product, the third extension product, a surrogate of an extension product, or combinations thereof.

25. The method of claim 24, wherein the detecting comprises quantitating the first extension product, the second extension product, the third extension product, the surrogate of an extension product, or combinations thereof.

26. The method of claim 17, wherein the target-complementary portion of the first primer comprises a nucleotide analog, the first extension product-complementary portion of the second primer comprises a nucleotide analog, or the target-complementary portion of the first primer and the first extension product-complementary portion of the second primer each comprise a nucleotide analog.

27. The method of claim 26, wherein the target-complementary portion of the first primer comprises a multiplicity of nucleotide analogs, the first extension product-complementary portion of the second primer comprises a multiplicity of nucleotide analogs, or the target-complementary portion of the first primer and the first extension product-complementary portion of the second primer each comprise a multiplicity of nucleotide analogs.

28. The method of claim 26, wherein the nucleotide analog comprises a 5-methylcytosine, a 2-amino-dA, a C-5 propynyl-dc, a C-5 propynyl-dU, a LNA, a 2′-O-methyl nucleotide, a phosphoroamidate nucleotide, or combinations thereof.

29. The method of claim 26, further comprising detecting the first extension product, the second extension product, the third extension product, a surrogate of an extension product, or combinations thereof.

30. The method of claim 29, wherein the detecting comprises quantitating the first extension product, the second extension product, the third extension product, the surrogate of an extension product, or combinations thereof.

31. A method for reducing strand amplification bias with bisulfite treated gDNA comprising:
(a) annealing a first primer with the bisulfite treated gDNA at a first annealing temperature, wherein the first primer comprises: (i) a target-complementary portion and (ii) a first primer-binding site upstream from the target-complementary portion;
(b) extending the annealed first primer to generate a first extension product;
(c) annealing a second primer with the first extension product at a second annealing temperature, wherein the second primer comprises: (i) a first extension product-complementary portion and (ii) a second primer-binding site upstream from the first extension product-complementary portion;
(d) extending the annealed second primer to generate a second extension product;
(e) annealing a third primer with the complement of the first primer-binding site of the second extension product at a third annealing temperature;
(f) extending the annealed third primer to generate a third extension product;
(g) annealing a fourth primer with the complement of the second primer-binding site of the third extension product at a fourth annealing temperature;
(h) extending the annealed fourth primer to generate an additional second extension product; and
(i) optionally, repeating steps (e)-(h) at least one additional cycle.

32. The method of claim 31, wherein the first annealing temperature, the second annealing temperature, the third annealing temperature, and the fourth annealing temperature are the same or substantially the same.

33. The method of claim 31, wherein the first annealing temperature is at least five ° C. less than the third annealing temperature.

34. The method of claim 33, wherein the first annealing temperature is at least ten ° C. less than the third annealing temperature

35. The method of claim 33, wherein (i) the first annealing temperature and the second annealing temperature are the same or substantially the same and (ii) the third annealing temperature and the fourth annealing temperature are the same or substantially the same.

36. The method of claim 31, further comprising detecting the first extension product, the second extension product, the third extension product, a surrogate of an extension product, or combinations thereof.

37. The method of claim 36, wherein the detecting comprises quantitating the first extension product, the second extension product, the third extension product, the surrogate of an extension product, or combinations thereof.

38. A method for reducing strand amplification bias with bisulfite treated gDNA comprising:
(a) annealing a first primer with the bisulfite treated gDNA at a first annealing temperature, wherein the first primer comprises: (i) a target-complementary portion comprising a nucleotide analog and (ii) a first primer-binding site upstream from the target-complementary portion;
(b) extending the annealed first primer to generate a first extension product;
(c) annealing a second primer with the first extension product at a second annealing temperature, wherein the second primer comprises: (i) a first extension product-complementary portion comprising a nucleotide analog and (ii) a second primer-binding site upstream from the first extension product-complementary portion;
(d) extending the annealed second primer to generate a second extension product;
(e) annealing a third primer with the complement of the first primer-binding site of the second extension product at a third annealing temperature;
(f) extending the annealed third primer to generate a third extension product;
(g) annealing a fourth primer with the complement of the second primer-binding site of the third extension product at a fourth annealing temperature;
(h) extending the annealed fourth primer to generate an additional second extension product; and
(i) optionally, repeating steps (e)-(h) at least one additional cycle.

39. The method of claim 38, wherein the first annealing temperature, the second annealing temperature, the third annealing temperature, and the fourth annealing temperature are the same or substantially the same.

40. The method of claim 38, wherein the first annealing temperature is at least five ° C. less than the third annealing temperature.

41. The method of claim 40, wherein the first annealing temperature is at least ten ° C. less than the third annealing temperature

42. The method of claim 40, wherein (i) the first annealing temperature and the second annealing temperature are the same or substantially the same and (ii) the third annealing temperature and the fourth annealing temperature are the same or substantially the same.

43. The method of claim 38, wherein the target-complementary portion of the first primer comprises a multiplicity of nucleotide analogs, the first extension product-complementary portion of the second primer comprises a multiplicity of nucleotide analogs, or the target-complementary portion of the first primer and the first extension product-complementary portion of the second primer each comprise a multiplicity of nucleotide analogs.

44. The method of claim 38, wherein the nucleotide analog comprises a 5-methylcytosine, a 2-amino-dA, a C-5 propynyl-dC, a C-5 propynyl-dU, a LNA, a 2′-O-methyl nucleotide, a phosphoroamidate nucleotide, or combinations thereof.

45. The method of claim 38, further comprising detecting the first extension product, the second extension product, the third extension product, a surrogate of an extension product, or combinations thereof.

46. The method of claim 45, wherein the detecting comprises quantitating the first extension product, the second extension product, the third extension product, the surrogate of an extension product, or combinations thereof.

47. A method for reducing strand amplification bias with bisulfite treated gDNA comprising:
(a) annealing a first primer with the bisulfite treated gDNA at a first annealing temperature, wherein the first primer comprises: (i) a target-complementary portion and (ii) a first primer-binding site upstream from the target-complementary portion;
(b) extending the annealed first primer to generate a first extension product comprising a nucleotide analog;
(c) annealing a second primer with the first extension product at a second annealing temperature, wherein the second primer comprises: (i) a first extension product-complementary portion and (ii) a second primer-binding site upstream from the first extension product-complementary portion;
(d) extending the annealed second primer to generate a second extension product comprising a nucleotide analog;
(e) annealing a third primer with the complement of the first primer-binding site of the second extension product at a third annealing temperature;
(f) extending the annealed third primer to generate a third extension product;
(g) annealing a fourth primer with the complement of the second primer-binding site of the third extension product at a fourth annealing temperature;
(h) extending the annealed fourth primer to generate an additional second extension product; and
(i) optionally, repeating steps (e)-(h) at least one additional cycle.

48. The method of claim 47, wherein the first annealing temperature, the second annealing temperature, the third annealing temperature, and the fourth annealing temperature are the same or substantially the same.

49. The method of claim 47, wherein the first annealing temperature is at least five ° C. less than the third annealing temperature.

50. The method of claim 49, wherein the first annealing temperature is at least ten ° C. less than the third annealing temperature

51. The method of claim 50, wherein (i) the first annealing temperature and the second annealing temperature are the same or substantially the same and (ii) the third annealing temperature and the fourth annealing temperature are the same or substantially the same.

52. The method of claim 47, wherein the first extension product comprises a multiplicity of nucleotide analogs, the second extension product comprises a multiplicity of nucleotide analogs, or the first extension product and the second extension product each comprise a multiplicity of nucleotide analogs.

53. The method of claim 47, wherein the nucleotide analog comprises a 5-methylcytosine, a 2-amino-dA, a C-5 propynyl-dc, a C-5 propynyl-dU, a LNA, a 2′-O-methyl nucleotide, a phosphoroamidate nucleotide, or combinations thereof.

54. The method of claim 47, further comprising detecting the first extension product, the second extension product, the third extension product, a surrogate of an extension product, or combinations thereof.

55. The method of claim 54, wherein the detecting comprises quantitating the first extension product, the second extension product, the third extension product, the surrogate of an extension product, or combinations thereof.

56. The method of claim 47, wherein the target-complementary portion of the first primer comprises a nucleotide analog, the first extension product-complementary portion of the second primer comprises a nucleotide analog, or the target-complementary portion of the first primer and the first extension product-complementary portion of the second primer each comprise a nucleotide analog.

57. The method of claim 56, wherein the target-complementary portion of the first primer comprises a multiplicity of nucleotide analogs, the first extension product-complementary portion of the second primer comprises a multiplicity of nucleotide analogs, or the target-complementary portion of the first primer and the first extension product-complementary portion of the second primer each comprise a multiplicity of nucleotide analogs.

58. The method of claim 56, wherein the nucleotide analog comprises a 5-methylcytosine, a 2-amino-dA, a C-5 propynyl-dc, a C-5 propynyl-dU, a LNA, a 2′-O-methyl nucleotide, a phosphoroamidate nucleotide, or combinations thereof.

59. The method of claim 56, further comprising detecting the first extension product, the second extension product, the third extension product, a surrogate of an extension product, or combinations thereof.

60. The method of claim 59, wherein the detecting comprises quantitating the first extension product, the second extension product, the third extension product, the surrogate of an extension product, or combinations thereof.

61. A method of reducing bias in a PCR amplification, wherein at least two versions of a target genomic locus are present, wherein the two versions differ in their nucleotide composition, the method comprising generating a first extension product, a second extension product, a third extension product, or combinations thereof, in a reaction composition comprising (a) a nucleotide analog and (b) a tailed first primer, a tailed second primer, or a tailed first primer pair.

62. The method of claim 61, wherein the genomic locus is not treated with sodium bisulfite.

63. The method of claim 62, wherein the genomic locus comprises a multiplicity of SNP sites, wherein at least two SNP sites comprise an A or T in one SNP allele and a C or G in the corresponding SNP allele.

64. The method of claim 63, further comprising detecting the first extension product, the second extension product, the third extension product, a surrogate of an extension product, or combinations thereof.

65. The method of claim 64, wherein the detecting comprises quantitating the first extension product, the second extension product, the third extension product, the surrogate of an extension product, or combinations thereof.

66. A kit comprising sodium bisulfite, a polymerase, and a tailed primer pair comprising (1) a first primer comprising (i) a target-complementary portion and (ii) a tail comprising a first primer-binding site upstream from the target-complementary portion and (2), and a second primer comprising (i) a first extension product-complementary portion and (ii) a tail comprising a second primer-binding site upstream from the first extension product-complementary portion.

67. The kit of claim 66, further comprising a nucleotide analog.

68. The kit of claim 66, wherein the target-complementary portion of the first primer comprises a nucleotide analog, the first extension product-complementary portion of the second primer comprises a nucleotide analog, or target-complementary portion of the first primer and the first extension product-complementary portion of the second primer each comprise a nucleotide analog.

69. The kit of claim 68, further comprising a third primer and a fourth primer.


SEQUENCE LISTING (13 sequences)

SEQ ID NO: 1 - DNA, 38 bp, Artificial Sequence
  Synthetic DNA
caggaaacagctatgaccctacacccaaatttccatta

SEQ ID NO: 2 - DNA, 42 bp, Artificial Sequence
  Synthetic DNA
tgtaaaacgacggccagttagtttaatgagtttaggtttttt

SEQ ID NO: 3 - DNA, 26 bp, Artificial Sequence
  Synthetic DNA
aacaaactaaataaccaatccaaaac

SEQ ID NO: 4 - DNA, 25 bp, Artificial Sequence
  Synthetic DNA
ttagagtagagggtgaaggtttttt

SEQ ID NO: 5 - DNA, 44 bp, Artificial Sequence
  Synthetic DNA
caggaaacagctatgaccaacaaactaaataaccaatccaaaac

SEQ ID NO: 6 - DNA, 43 bp, Artificial Sequence
  Synthetic DNA
tgtaaaacgacggccagtttagagtagagggtgaaggtttttt

SEQ ID NO: 7 - DNA, 18 bp, Artificial Sequence
  Synthetic DNA
caggaaacagctatgacc

SEQ ID NO: 8 - DNA, 18 bp, Artificial Sequence
  Synthetic DNA
tgtaaaacgacggccagt

SEQ ID NO: 9 - DNA, 38 bp, Artificial Sequence; identical to SEQ ID NO: 1
  Synthetic DNA

SEQ ID NO: 10 - DNA, 42 bp, Artificial Sequence; identical to SEQ ID NO: 2
  Synthetic DNA

SEQ ID NO: 11 - DNA, 38 bp, Artificial Sequence; identical to SEQ ID NO: 1
  Synthetic DNA

SEQ ID NO: 12 - DNA, 42 bp, Artificial Sequence; identical to SEQ ID NO: 2
  Synthetic DNA

SEQ ID NO: 13 - DNA, 187 bp, Artificial Sequence
  Synthetic DNA
tagtttaatgagtttaggttttttcgatatggttcggttgggttcgtgtttcgttggttttgggcgttagtaagcgcgggtcgggcggggttatagggcgggtttcgattttagcgttttttttaggatttagattgggcggcgggaaggagttgaggagagtcgcgtaatggaaatttgggtgtag